(19) United States
(12) Patent Application Publication
(10) Pub. No.: US 2022/0160245 A1
(43) Pub. Date: May 26, 2022

(54) SYSTEM AND METHOD OF DETERMINING DISEASE BASED ON HEAT MAP IMAGE EXPLAINABLE FROM ELECTROCARDIOGRAM SIGNAL

(71) Applicant: Industry- Academic Cooperation Foundation, Chosun University, Gwangju (KR)

(72) Inventors: Keun Chang KWAK, Gwangju (KR); Yeong Hyun BYEON, Jangeog-gun (KR); Chan Uk YEOM, Gwangju (KR); Myung Won LEE, Gwangju (KR)

(21) Appl. No.: 17/535,617

(22) Filed: Nov. 25, 2021

(30) Foreign Application Priority Data
Nov. 26, 2020 (KR) 10-2020-0161074

(51) Int. Cl. A61B 5/0205; A61B 5/01; A61B 5/318; A61B 5/00

(52) U.S. Cl. CPC A61B 5/02055; A61B 5/015; A61B 5/318; A61B 5/7203; A61B 5/742; A61B 5/726; A61B 5/7267; A61B 5/7221

(57) ABSTRACT

The present invention relates to a system and a method of determining disease based on a heat map image explainable from an electrocardiogram signal, which determine an electrocardiogram signal as a normal signal and a disease signal by transfer-learning a transfer-learning model through a deep learning network, calculate and visualize a part with a high relevance score to the determination to enable a user to objectively and finally determine the disease and the normal state. The system for determining disease based on a heat map image explainable from an electrocardiogram signal includes: an electrocardiogram measuring unit configured to acquire an electrocardiogram signal; a scalogram transform unit configured to transform the electrocardiogram signal acquired from the electrocardiogram measuring unit into a time-frequency region and store the transformed electrocardiogram signal as a two-dimensional image; a disease determining unit configured to determine the electrocardiogram signal as normal/disease through the two-dimensional image stored in the scalogram transform unit; a relevance score calculating unit configured to calculate a part contributed to determination of the electrocardiogram signal as normal/disease by the disease determining unit; and a heat map display unit configured to display the part contributed to the determination of the electrocardiogram signal as normal/disease calculated by the relevance score calculating unit as a heat map.

### CROSS-REFERENCE TO RELATED APPLICATIONS

This application claims priority to and the benefit of Korean Patent Application No. 10-2020-0161074 filed on Nov. 26, 2020, which is incorporated herein by reference in its entirety.

### TECHNICAL FIELD

The present invention relates to a system and a method of determining disease based on a heat map image explainable from an electrocardiogram signal, and more particularly, to a system and a method of determining disease based on a heat map image explainable from an electrocardiogram signal, which determine an electrocardiogram signal as a normal signal and a disease signal by transfer-learning a transfer-learning model through a deep learning network, calculate and visualize a part with a high relevance score to the determination to enable a user to objectively and finally determine the disease and the normal state.

### BACKGROUND ART

According to the WHO, cardiovascular diseases (CVDs) are the leading cause of death today. An electrocardiogram is a non-invasive medical tool that displays the heart rhythm and condition, and refers to the analysis of the electrical activity of the heart and the record of the result of the analysis in the form of wavelengths.

The analysis of the electrocardiogram is essentially used for diagnosing heart disease, especially, cardiac arrhythmias with irregular heartbeats. Further, in addition to arrhythmias, the analysis of the electrocardiogram is useful for diagnosing myocardial disorders, atrial ventricle hypertrophy, dilatation, pulmonary circulation disorders, electrolyte metabolism abnormalities, drug effect confirmation, and other heart diseases and related diseases. Therefore, automatic detection of irregular heart rhythms in ECG signals is very important in the field of cardiology.

Recently, electrocardiogram-based personal identification, disease classification, emotion recognition, etc. are used by using a deep learning network, which is sometimes possible to classify disease more accurately than humans.

However, in the related art, when a heart-related disease is classified with a dimensional electrocardiogram signal and visualized by applying artificial intelligence that can be dimensionally explained, it is difficult to determine which signal the deep learning network determines and classifies the electrocardiogram signal based on.

### SUMMARY OF THE INVENTION

The present invention has been made in an effort to solve the problems in the related art, and provides a system and a method of determining disease based on a heat map image explainable from an electrocardiogram signal, which determine an electrocardiogram signal as a normal signal and a disease signal by transfer-learning a transfer-learning model through a deep learning network, calculate and visualize a part with a high relevance score for the determination to enable a user to objectively and finally determine the disease and the normal state.

An exemplary embodiment of the present invention provides a system for determining disease based on a heat map image explainable from an electrocardiogram signal includes: an electrocardiogram measuring unit configured to acquire an electrocardiogram signal; a scalogram transform unit configured to transform the electrocardiogram signal acquired from the electrocardiogram measuring unit into a time-frequency region and store the transformed electrocardiogram signal as a two-dimensional image; a disease determining unit configured to determine the electrocardiogram signal as normal/disease through the two-dimensional image stored in the scalogram transform unit; a relevance score calculating unit configured to calculate a part contributed to determination of the electrocardiogram signal as normal/disease by the disease determining unit; and a heat map display unit configured to display the part contributed to the determination of the electrocardiogram signal as normal/disease calculated by the relevance score calculating unit as a heat map.

The heat map display unit may include a heat map visualizing unit which displays the part contributed to the determination of the electrocardiogram signal as normal/disease in the electrocardiogram signal.

The disease determining unit may be a transfer-learning model which is transfer-trained with a deep learning network so as to determine the electrocardiogram signal as normal/disease through the plurality of two-dimensional images.

The electrocardiogram signal may be acquired by a sensor, and has a one-dimensional vector form.

When the electrocardiogram signal is determined as an abnormal signal and disease, the relevance score calculating unit may calculate a relevance score of the abnormal signal by using a Layer-wise Relevance Propagation (LRP) method.

The scalogram transform unit may divide the electrocardiogram signal into a normal signal scalogram in which the electrocardiogram signal is normal and a disease signal scalogram in which the electrocardiogram signal is abnormal and store the divided electrocardiogram signals as the two-dimensional images.

The electrocardiogram signal may pass through a low-band pass filter and a high-band pass filter in order to remove noise included in an original signal acquired by the sensor.

When the heat map display unit displays a part contributed to the determination of the disease, the part closer to red may have a higher relevance score.

Another exemplary embodiment of the present invention provides a method of determining disease based on a heat map image explainable from an electrocardiogram signal, the method including: an electrocardiogram measuring operation of acquiring an electrocardiogram signal; a noise removing operation of removing noise from the acquired electrocardiogram signal; a scalogram transforming operation of converting the electrocardiogram signal into a time-frequency region and storing the converted electrocardiogram signal as a two-dimensional image; a disease determining operation of determining the electrocardiogram signal as normal/disease through the two-dimensional image; a relevance score calculating operation of calculating a part contributed to binary classification of the electrocardiogram signal into normal/disease; a heat map displaying operation of displaying the part contributed to the determination of the electrocardiogram signal as normal/disease as a heat map; and a contributed part displaying operation of displaying the part contributed to the heat map in the electrocardiogram signal.

The system and the method of determining disease based on a heat map image explainable from an electrocardiogram signal according to the present invention may determine a normal signal and a disease signal of an electrocardiogram signal by transfer-learning a transfer-learning model through a deep learning network, calculate and visualize a part with a high relevance score to the determination, thereby achieving an effect that a user is capable of objectively and finally determining disease and a normal state.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram illustrating a configuration of a system for determining disease based on a heat map image explainable from an electrocardiogram signal according to the present invention.

FIG. 2 is a diagram illustrating the case where a processing signal is generated by removing noise from an electrocardiogram original signal by the system for determining disease based on a heat map image explainable from an electrocardiogram signal according to the present invention.

FIG. 3 is a diagram illustrating a scalogram transform of the system for determining disease based on a heat map image explainable from an electrocardiogram signal according to the present invention.

FIG. 4 is a diagram illustrating determination of a disease by a disease determining unit of the system for determining disease based on a heat map image explainable from an electrocardiogram signal according to the present invention.

FIG. 5 is a diagram illustrating calculation of a part of the electrocardiogram processing signal contributed to the determination of the normal state/disease by using an LRP method by a relevance score calculating unit of the system for determining disease based on a heat map image explainable from an electrocardiogram signal according to the present invention.

FIGS. 6 and 7 are diagrams illustrating the part contributed to the determination of the disease by using the LRP method by a heat map by a heat map display unit of the system for determining disease based on a heat map image explainable from an electrocardiogram signal according to the present invention.

FIGS. 8 and 9 are diagrams illustrating a method of determining disease based on a heat map image explainable from an electrocardiogram signal according to the present invention.

### DETAILED DESCRIPTION

Hereinafter, a system 10 and a method of determining disease based on a heat map image explainable from an electrocardiogram signal according to an exemplary embodiment of the present invention will be described with reference to the accompanying drawings. The present invention may have various modifications and various forms and thus specific exemplary embodiments will be illustrated in the drawings and described in detail in the context. However, it is not intended to limit the present invention to the specific disclosed form, and it will be appreciated that the present invention includes all modifications, equivalences, or substitutions included in the spirit and the technical scope of the present invention. In describing each drawing, like reference numerals in the drawings refer to the same or similar functions. In the drawings, the thickness of layers, films, panels, regions, etc., are exaggerated for clarity of the present invention.

Terms, such as first and second, may be used for describing various constituent elements, but the constituent elements are not limited by the terms. The terms are used only to discriminate one constituent element from another constituent element. For example, without departing from the scope of the invention, a first constituent element may be named as a second constituent element, and similarly a second constituent element may be named as a first constituent element.

Terms used in the present application are used only to describe specific exemplary embodiments, and are not intended to limit the present invention. Singular expressions used herein include plurals expressions unless they have definitely opposite meanings in the context. In the present application, it will be appreciated that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance.

All terms used herein including technical or scientific terms have the same meanings as meanings which are generally understood by those skilled in the art unless they are differently defined. Terms defined in generally used dictionary shall be construed that they have meanings matching those in the context of a related art, and shall not be construed in ideal or excessively formal meanings unless they are clearly defined in the present application.

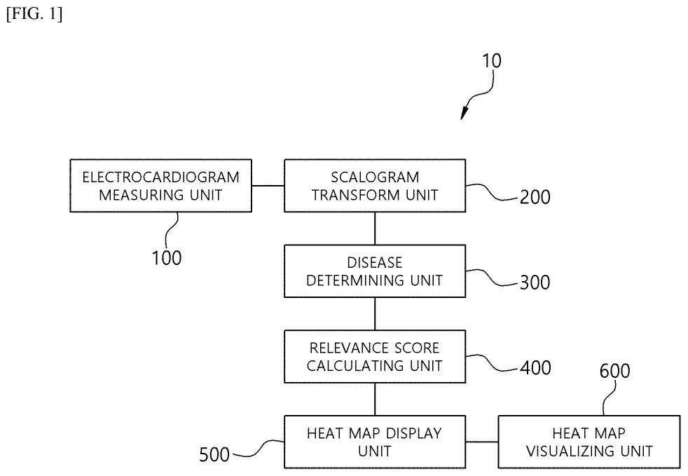

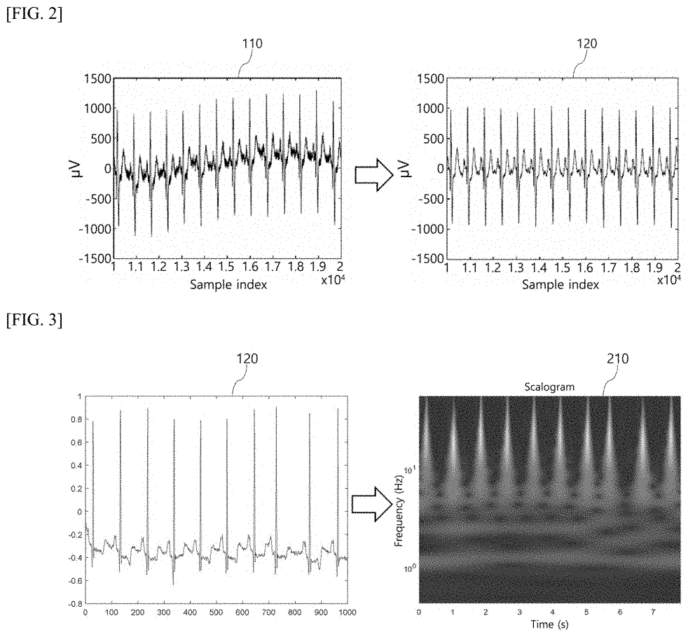

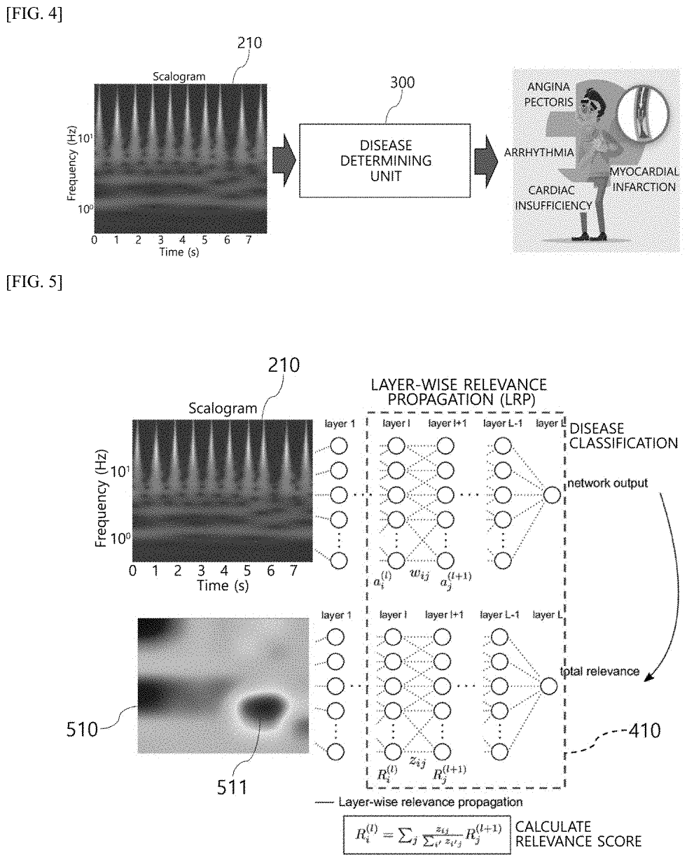

FIGS. 1 to 7 illustrate an exemplary embodiment of a system 10 for determining disease based on a heat map image explainable from an electrocardiogram signal according to the present invention.

Referring to the drawings, the system 10 for determining disease based on a heat map image explainable from an electrocardiogram signal includes an electrocardiogram measuring unit 100, a scalogram transforming unit 200, a disease determining unit 300, a relevance score calculating unit 400, and a heat map display unit 500.

The electrocardiogram measuring unit 100 acquires an electrocardiogram original signal 110. The electrocardiogram original signal 110 is acquired from a sensor attached to a body, and has a one-dimensional vector form.

The electrocardiogram measuring unit 100 removes noise by making the electrocardiogram original signal 110 acquired by the sensor pass through a low-band pass filter and a high-band pass filter and processes the electrocardiogram original signal 110 to an electrocardiogram processed signal 120. In this case, the noise refers to all sounds except for the electrocardiogram original signal 110.

The low-band pass filter and the high-band pass filter uses a one-dimensional convolution operation.

In the present exemplary embodiment, an average filter of 500 MHz is used in the low-band pass filter, and an average filter of 10 MHz is used in the high-band pass filter, but the present invention is not limited thereto, and a user may also adjust the range of the filter.

Further, a detailed length of the filter may be adjusted according to a sampling frequency of the signal.

The scalogram transforming unit 200 transforms the electrocardiogram processed signal 120 acquired from the electrocardiogram measuring unit 100 into a time-frequency region, and divides a normal signal scalogram in which electrocardiogram processed signal 120 is normal and a disease signal scalogram in which the electrocardiogram processed signal 120 is abnormal and stores the scalograms signals in the form of a two-dimensional image.

The scalogram transforming unit 200 uses a wavelet transform that is one of the signal processing methods that visualize a one-dimensional signal. The wavelet transform is a time-frequency transform, and since the horizontal axis is the time axis and the vertical axis is the frequency axis, waveform information does not appear like a spectrogram, and the change over time for each frequency band may be visually grasped.

The scalogram transforming unit 200 is an absolute value of a continuous wavelet transform efficient, and decomposes the wavelet signal into enlarged or shifted ones of a mother wavelet.

Ψ that is a basic wavelet is referred to as a mother wavelet, and a shifted and enlarged one is referred to as a daughter wavelet, “a” is a scale factor, and “b” is a shift factor. When R is a real number, a is a non-zero positive real number (R⁺−0), and when b is a real number and f(t) is an original signal, a continuous wavelet transform formula of the electrocardiogram processed signal 120 is as represented below.

$$CWT ( a , b ) = \frac{1}{\sqrt{a}} {\int }_{- ∞}^{∞} f ( t ) * ψ ( \frac{t - b}{a} ) d t \\ a ∈ {R}^{+} - 0 , b ∈ R \qquad [ Equation 1 ]$$

The disease determining unit 300 determines the electrocardiogram processed signal 120 as normal or disease through a two-dimensional image 210.

The disease determining unit 300 uses a transfer learning model that is transfer-trained through a Convolutional Neural (CNN)-based deep neural network so as to binary-classify the electrocardiogram processed signal 120 into normal and disease through the two-dimensional image 210.

In this case, as the transfer learning model, models validated by researchers or companies may be used, and in the present exemplary embodiment, a skip connection-trained ResNet model is applied, but in addition to this, other published models, such as Googlenet, may also be used, and the deep-learning network training may also be directly performed.

When the electrocardiogram processed signal 120 is determined as an abnormal signal 121, that is, disease, the relevance score calculating unit 400 calculates a relevance score of the abnormal signal 121 by using a Layer-wise Relevance Propagation (LRP) method 410.

The disease includes all of the diseases, such as angina pectoris, arrhythmia, cardiac insufficiency, and myocardial infarction, which are derivable from an electrocardiogram examination.

FIG. 5 illustrates a concept of calculating, by the relevance score calculating unit 400, a part contributed to the determination of the electrocardiogram processed signal 120 as normal/disease by using the LRP method 410.

Referring to the drawing, in the LRP 410, the relevance score represents the degree of a change in an output according to a change in an input.

In order to obtain the output, all of the relevance scores in neurons of a previous layer are added to calculate an output score, and the present invention is the process of reversely decomposing the output score. The relevance score may be decomposed as follows by the Taylor series.

$$f ( x ) = {\sum }_{i} {R}_{i} \qquad [ Equation 2 ]$$

When it is defined that an output of the neuron is f(x), a relevance score of the neural at an output end is R, and a sum of numerical progression is E, and when the relevance score and the neuron x are set to be the same and propagate to the previous layers, the relevance scores of all of the neurons may be calculated.

The heat map display unit 500 further includes a heat map visualizing unit 600.

The heat map display unit 500 display a part contributed to the determination of the electrocardiogram processed signal 120 as normal/disease by the relevance score calculating unit 400 as a heat map 510. In this case, the heat map 510 may be various forms of map, and is not limited in the form.

The heat map visualizing unit 600 displays the part 121 contributed to the determination of the electrocardiogram processed signal 120 as normal/disease in the electrocardiogram processed signal 120.

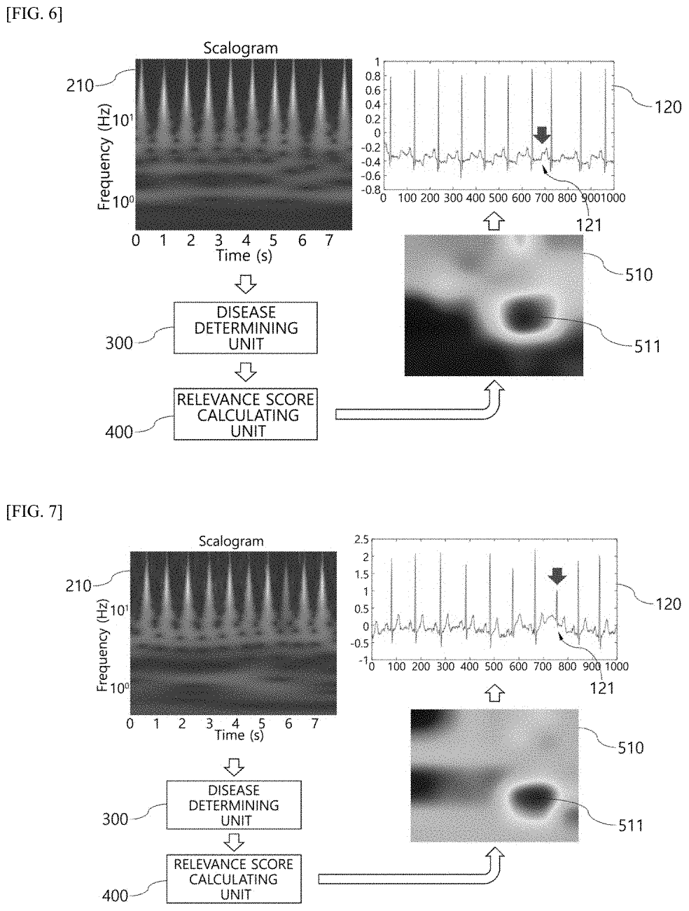

FIGS. 6 and 7 illustrate the concept of displaying, by the heat map display unit 500, the part contributed to the determination of the disease as the heat map 510 by using the LRP 410.

Referring to the drawings, the scalogram transform unit 200 transforms the electrocardiogram processed signal 120 into the two-dimensional image 210, the disease determining unit 300 determines the electrocardiogram processed signal 120 as normal/disease, and when the abnormal signal 121 is included in the electrocardiogram processed signal 120, so that the electrocardiogram processed signal 120 is determined as disease, the relevance score calculating unit 400 calculates a relevance score of the abnormal signal 121 by using the LRP method, and the heat map display unit 500 displays a calculation value as the heat map 510. In this case, when the heat map display unit 500 displays a part 511 contributed to the determination of the disease in the heat map 510, the part closer to red has a higher relevance score. Next, the heat map visualizing unit 600 visualizes the abnormal signal 121 displayed as the heat map 510 in the electrocardiogram processed signal 120, and the abnormal signal 121 may be the accurate basis for determining the disease by a user.

In FIGS. 6 and 7, it can be seen that the red regions appear differently in the heat map 510. This is the part 511 contributed to the determination of the disease by the relevance score calculating unit 400, and it can be confirmed that the disease is different. Further, the part 511 may be visualized in the electrocardiogram processed signal 120 by the heat map visualizing unit 600 to determine which electrocardiogram signal is an abnormal signal 121. Therefore, a user may visually check that the disease is different, so that the abnormal signal 121 may be the basis of the accurate determination.

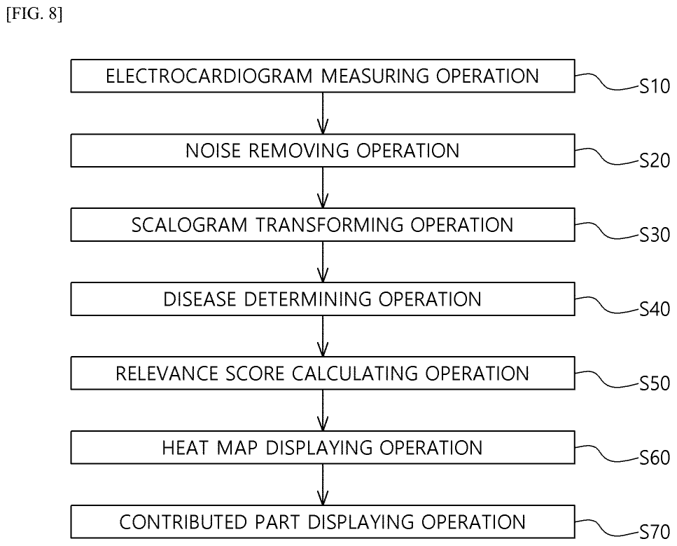

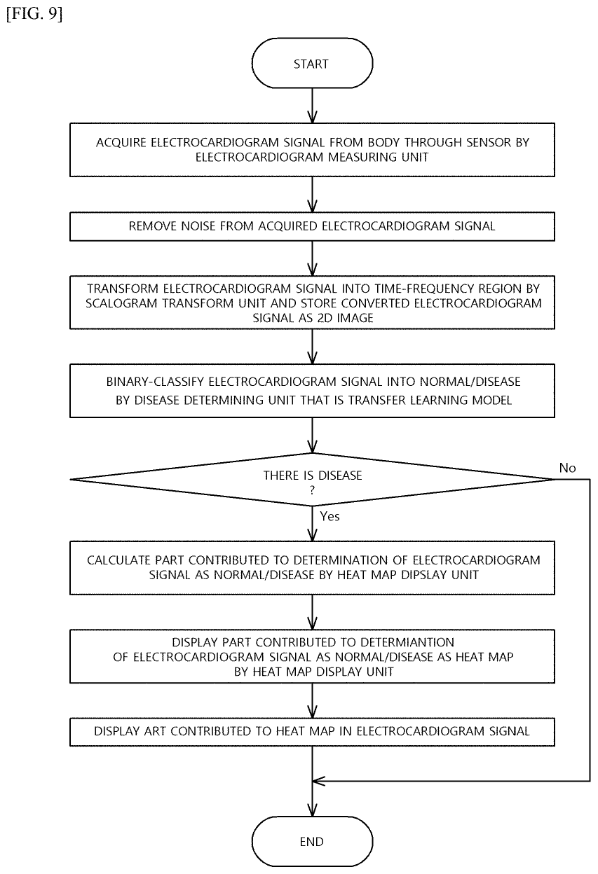

FIGS. 8 and 9 are diagrams illustrating a method of determining disease based on a heat map image explainable from an electrocardiogram signal according to the present invention.

Referring to the drawings, the method of determining disease based on a heat map image explainable from an electrocardiogram signal includes an electrocardiogram measuring operation S10 of acquiring an electrocardiogram original signal 110, a noise removing operation S20 of removing noise from the acquired electrocardiogram original signal 110, a scalogram transforming operation S30 of transforming an electrocardiogram signal into a time-frequency region and storing the transformed signal as a two-dimensional image, a disease determining operation S40 of classifying the electrocardiogram signal into normal/disease through the plurality of two-dimensional images, a relevance score calculating operation S50 of calculating a part contributed to binary classification of the electrocardiogram processed signal 120 into normal/disease, a heat map displaying operation S60 of displaying the part contributed to the determination of the electrocardiogram processed signal 120 as normal/disease as a heat map 510, and a contributed part displaying operation S70 of displaying the part contributed to the heat map 510 in the electrocardiogram processed signal 120.

The system 10 and the method of determining disease based on a heat map image explainable from an electrocardiogram signal determine an electrocardiogram signal as a normal signal and a disease signal by transfer-training a transfer learning model through a deep learning network, calculate and visualize a part of the electrocardiogram signal having a high relevance score for the determination, and enable a user to finally determine the disease and the normal state objectively.

The description of the presented exemplary embodiments is provided to enable those skilled in the art to use or carry out the present invention. Various modifications of the exemplary embodiments may be apparent to those skilled in the art, and general principles defined herein may be applied to other exemplary embodiments without departing from the scope of the present invention. Accordingly, the present disclosure is not limited to the exemplary embodiments suggested herein, and shall be interpreted within the broadest meaning range consistent to the principles and new characteristics presented herein.

What is claimed is:

1. A system for determining disease based on a heat map image explainable from an electrocardiogram signal, the system comprising:
an electrocardiogram measuring unit configured to acquire an electrocardiogram signal;
a scalogram transform unit configured to transform the electrocardiogram signal acquired from the electrocardiogram measuring unit into a time-frequency region and store the transformed electrocardiogram signal as a two-dimensional image;
a disease determining unit configured to determine the electrocardiogram signal as normal/disease through the two-dimensional image stored in the scalogram transform unit;
a relevance score calculating unit configured to calculate a part contributed to determination of the electrocardiogram signal as normal/disease by the disease determining unit; and
a heat map display unit configured to display the part contributed to the determination of the electrocardiogram signal as normal/disease calculated by the relevance score calculating unit as a heat map.

2. The system of claim 1, wherein the heat map display unit includes a heat map visualizing unit which displays the part contributed to the determination of the electrocardiogram signal as normal/disease in the electrocardiogram signal.

3. The system of claim 1, wherein the disease determining unit is a transfer-learning model which is transfer-trained with a deep learning network so as to determine the electrocardiogram signal as normal/disease through the plurality of two-dimensional images.

4. The system of claim 1, wherein the electrocardiogram signal is acquired by a sensor, and has a one-dimensional vector form.

5. The system of claim 1, wherein when the electrocardiogram signal is determined as an abnormal signal and disease, the relevance score calculating unit calculates a relevance score of the abnormal signal by using a Layer-wise Relevance Propagation (LRP) method.

6. The system of claim 1, wherein the scalogram transform unit divides the electrocardiogram signal into a normal signal scalogram in which the electrocardiogram signal is normal and a disease signal scalogram in which the electrocardiogram signal is abnormal and stores the divided electrocardiogram as the two-dimensional images.

7. The system of claim 1, wherein the electrocardiogram signal passes through a low-band pass filter and a high-band pass filter in order to remove noise included in an original signal acquired by the sensor.

8. The system of claim 1, wherein when the heat map display unit displays a part contributed to the determination of the disease, the part closer to red has a higher relevance score.

9. A method of determining disease based on a heat map image explainable from an electrocardiogram signal, the method comprising:
an electrocardiogram measuring operation of acquiring an electrocardiogram signal;
a noise removing operation of removing noise from the acquired electrocardiogram signal;
a scalogram transforming operation of converting the electrocardiogram signal into a time-frequency region and storing the converted electrocardiogram signal as a two-dimensional image;
a disease determining operation of determining the electrocardiogram signal as normal/disease through the two-dimensional image;
a relevance score calculating operation of calculating a part contributed to binary classification of the electrocardiogram signal into normal/disease;
a heat map displaying operation of displaying the part contributed to the determination of the electrocardiogram signal as normal/disease as a heat map; and
a contributed part displaying operation of displaying the part contributed to the heat map in the electrocardiogram signal.
